(19) United States
(12) Patent Application Publication
(10) Pub. No.: US 2023/0149362 A1
(43) Pub. Date: May 18, 2023

(54) NEW CAPSULE COMPOSITION FOR PERORAL ADMINISTRATION

(71) Applicant: Vicore Pharma AB, Stockholm (SE)

(72) Inventors: Ola Camber, Stockholm (SE); Stefan Gruden, Uppsala (SE)

(21) Appl. No.: 17/920,558

(22) PCT Filed: Apr. 23, 2021
(86) PCT No.: PCT/GB2021/050992

§ 371 Date: Oct. 21, 2022

(30) Foreign Application Priority Data
Apr. 24, 2020 (GB) 2006081.0

(51) Int. Cl. A61K 31/4178; A61K 9/48

(52) U.S. Cl. CPC A61K 31/4178; A61K 9/4858; A61K 9/4825; A61K 9/4833; A61K 9/0053

(57) ABSTRACT

According to the invention there is provided a pharmaceutical dosage form that is suitable for peroral administration to the gastrointestinal tract, which dosage form comprises a pharmaceutical composition in the form of a heterogeneous mixture comprising solid particles of N-butyloxycarbonyl-3-(4-imidazol-1-ylmethylphenyl)-5-iso-butylthiophene-2-sulfonamide (C21), or a pharmaceutically-acceptable salt thereof, suspended in a pharmaceutically-acceptable, hydrophobic, lipid-based carrier in which C21 or salt thereof is essentially insoluble, which composition is contained within a capsule that is suitable for such peroral administration. Preferred carriers include triglycerides. Such dosage forms find utility in the treatment of lung diseases, such as idiopathic pulmonary fibrosis, sarcoidosis and respiratory virus-induced tissue damage.

### FIELD OF THE INVENTION

This invention relates to new pharmaceutical dosage forms, their use as medicaments and particularly to their administration to treat, inter alia, lung diseases, for example interstitial lung diseases.

### BACKGROUND AND PRIOR ART

Interstitial lung diseases (ILDs) are a group of lung diseases that affect the interstitium, characterized by tissue around alveoli becoming scarred and/or thickened, and so inhibiting the respiratory process.

ILDs are distinct from obstructive airway diseases (e.g. chronic obstructive airway disease (COPD) and asthma), which are typically characterized by narrowing (obstruction) of bronchi and/or bronchioles. ILDs may be caused by injury to the lungs, which triggers an abnormal healing response but, in some cases, these diseases have no known cause. ILDs can be triggered by chemicals (silicosis, asbestosis, certain drugs), infection (e.g. pneumonia) or other diseases (e.g. rheumatoid arthritis, systemic sclerosis, myositis, hypersensitivity pneumonitis or systemic lupus erythematosus).

The most common ILDs are idiopathic pulmonary fibrosis (IPF) and sarcoidosis, both of which are characterized by chronic inflammation and reduced lung function.

Sarcoidosis is a disease of unknown cause that is characterized by collections of inflammatory cells that form lumps (granulomas), often beginning in the lungs (as well as the skin and/or lymph nodes, although any organ can be affected). When sarcoidosis affects the lungs, symptoms include coughing, wheezing, shortness of breath, and/or chest pain.

Treatments for sarcoidosis are patient-specific. In most cases, symptomatic treatment with non-steroidal anti-inflammatory drugs (NSAIDs) is possible, but for those presenting lung symptoms, glucocorticoids (e.g. prednisone or prednisolone), antimetabolites and/or monoclonal anti-tumor necrosis factor antibodies are often employed.

IPF is a lung-disease of unknown cause that affects about 5 million people globally. It has no curative treatment options except, in rare cases, lung transplantation, resulting in a chronic, irreversible, progressive deterioration in lung function and, in most cases, leading to death within 2-5 years (median survival 2.5 to 3.5 years). While the overall prognosis is poor in IPF, it is difficult to predict the rate of progression in individual patients. Risk factors for IPF include age, male gender, genetic predisposition and history of cigarette smoking. The annual incidence is between 5-16 per 100,000 individuals, with a prevalence of 13-20 cases per 100,000 people, increasing dramatically with age (King Jr T E et al., Lancet (2011) 378, 1949-1961; Noble P W et al., J. Clin. Invest. (2012) 122, 2756-2762). IPF is limited to the lungs and is recalcitrant to therapies that target the immune system which distinguishes it from pulmonary fibrosis (PF) associated with systemic diseases.

Patients with IPF usually seek medical assistance clue to chronic and progressive exertional dyspnea and cough. Imaging of the lung classically reveals traction bronchiectasis, thickened interlobar septae and subpleural honeycombing. When all three manifestations are present and there is no evidence of a systemic connective tissue disease or environmental exposure, a diagnosis of IPF is very likely. A definite diagnosis is usually made by lung biopsy and requires a multidisciplinary team of expertise including pulmonologists, radiologists and pathologists experienced in ILDs.

IPF demonstrates different phenotypes with different prognosis, defined as mild, moderate and severe. Mild cases follow a stable or slow progressive path with patients sometimes taking several years to seek medical advice. Accelerated IPF has a much more rapid progression with shortened survival, affecting a sub-group of patients, usually male cigarette smokers. Acute exacerbations of IPF are defined as a rapid worsening of the disease, and patients in this sub-population have very poor outcomes with a high mortality rate in the short run. The cause of IPF is unknown but it appears to be a disorder likely arising from an interplay of environmental and genetic factors resulting in fibroblast driven unrelenting tissue remodeling rather than normal repair; a pathogenesis primarily driven by fibrosis rather than inflammation. A growing body of evidence suggests that the disease is initiated through alveolar epithelial cell microinjuries and apoptosis, activating neighboring epithelial cells and attracting stem or progenitor cells that produce the factors responsible for the expansion of the fibroblast and myofibroblast populations in a tumor like way. The fibroblastic foci secrete exaggerated amounts of extracellular matrix that destroys the lung parenchyma and ultimately leads to loss of lung function.

The mean annual rate of decline in lung function (vital capacity) is within a range of 0.13-0.21 litres. Symptoms precede diagnosis by 1-2 years and radiographic signs may precede symptoms (Ley B et al., Am. J. Respir. Crit. Care Med. (2011) 183, 431-440).

Numerous treatment approaches have been tested in pre-clinical models and clinical trials such as anti-inflammatory, immune-modulatory, cytotoxic, general anti-fibrotic, anti-oxidant, anti-coagulant, anti-chemokine, anti-angiogenic drugs as well as RAS-blockers, endothelin antagonists, and sildenafil, all of which have basically been shown to provide limited or no benefits (Rafii R et al., J. Thorac. Dis. (2013) 5, 48-73).

Current treatment of IPF includes oxygen supplementation. Medications that are used include pirfenidone or nintedanib, but only with limited success in slowing the progression of the disease. Further, both of these drugs commonly cause (predominantly gastrointestinal) side-effects.

There are drawbacks associated with all of the aforementioned ILD (and IPF) drug treatments and there is a real clinical need for safer and/or more effective treatments.

To restore the alveolar epithelium is very desirable as a therapeutic effect in IPF, and therefore stem cell therapy has also been tested. Some preclinical studies have shown promise in the use of pluripotent stem cells that can differentiate into lung epithelial and endothelial cells, thereby repairing lung injury and fibrosis.

Currently, a lung transplant is the only intervention that substantially improves survival in IPF patients. However, complications such as infections and transplant rejection are not uncommon.

The development of new treatment strategies for IPF is therefore important. Thus, the fundamental challenge for the future is to develop appropriate therapeutic approaches that will reverse or stop the progression of the disease.

The Renin-Angiotensin System (RAS) is a key regulator of blood pressure homeostasis. Renin, a protease, cleaves its only known substrate (angiotensinogen) to form angiotensin I (Ang I), which in turn serves as substrate to angiotensin converting enzyme (ACE) to form Ang II. The endogenous hormone Ang II is a linear octapeptide (Asp¹-Arg²-Val³-Tyr⁴-Ile⁵-His⁶-Pro⁷-Phe⁸) and is an active component of the renin angiotensin system (RAS).

The angiotensin II type 1 (AT1) receptor is expressed in most organs and is believed to be responsible for the majority of the pathological effects of Ang II. The safety and efficacy of losartan (an AT1-receptor inhibitor) has recently been investigated in a small uncontrolled open-label pilot trial on IPF (www.dinicaltrials.gov identifier NCT00879879).

Several studies in adult individuals appear to demonstrate that, in the modulation of the response following Ang II stimulation, activation of the angiotensin II type 1 (AT2) receptor has opposing effects to those mediated by the AT1 receptor.

The AT2 receptor has also been shown to be involved in apoptosis and inhibition of cell proliferation (de Gasparo M et al., Pharmacol. Rev., 2000; 52:415-472).

AT2 receptor agonists have also been shown to be of potential utility in the treatment and/or prophylaxis of disorders of the alimentary tract, such as dyspepsia and irritable bowel syndrome, as well as multiple organ failure (see international patent application WO 99/43339).

The expected pharmacological effects of agonism of the AT2 receptor are described in general in de Gasparo M et al., supra. It is not mentioned that agonism of the AT2 receptor may be used to treat IPF.

International patent application WO 2002/096883 describes the preparation of imidazolyl, triazolyl, and tetrazolyl thiophene sulfonamides and derivatives as AT2 receptor agonists. Of the compounds described in that document (as Example 1) is N-butyloxycarbonyl-3-(4-imidazol-1-ylmethylphenyl)-5-iso-butylthiophene-2-sulfonamide (Compound 21 or, as used hereinafter ‘C21’), which was selected for clinical development from a group of about 20 related analogues as a selective AT2 receptor agonist. C21 is now in clinical development for treatment of AT2 receptor related disorders in which treatment with an AT2 receptor agonist is believed to be beneficial, including IPF (see, for example, international patent application WO 2016/139475).

Formulative work carried out in respect of C21 and salts thereof has proven extremely difficult. Part of the issue is the hitherto unreported extreme sensitivity of C21 and salts thereof to the combined presence of light and water. Furthermore, attempts to provide stable solid state formulations, even in the dry state, have produced blends with conventional excipients that are chemically unstable. These pieces of information have not been made available to the public previously.

As a consequence, C21 has previously been formulated as an aqueous solution, which is frozen whilst stored and then thawed immediately prior to peroral dosing. Protecting C21 in this way from light-catalyzed aqueous decomposition presents logistic issues as far as shipping drug product around the world is concerned. A more stable, pharmaceutically-acceptable composition is highly desirable, if not a requirement, for a commercially-viable product.

The applicant has been working with this active ingredient for nearly 20 years, and, until recently, has not managed to obtain a pharmaceutically-acceptable dosage form, that is one in which the active ingredient is stable when stored at ambient temperatures, in a reproducible way.

In attempting to prepare such an improved peroral capsule-based dosage form, the applicant has found that it is possible to solve the above problems by suspending particles of C21, or a pharmaceutically-acceptable salt thereof, in certain specific carriers, as described hereinafter.

### DISCLOSURE OF THE INVENTION

According to a first aspect of the invention, there is provided a pharmaceutical dosage form that is suitable for peroral administration to the gastrointestinal tract, which dosage form comprises a pharmaceutical composition in the form of a heterogeneous mixture comprising solid particles of C21, or a pharmaceutically-acceptable salt thereof, suspended in a pharmaceutically-acceptable, hydrophobic, lipid-based carrier in which C21 or salt thereof is essentially insoluble, which composition is contained within a capsule that is suitable for such peroral administration. Such dosage forms are hereinafter referred to together as ‘the dosage forms of the invention’.

Dosage forms of the invention are suitable for peroral administration and delivery, as a complete dosage form, to the gastrointestinal tract. This means that a dosage form of the invention should be suitable for swallowing as a whole, complete dosage form for subsequent consumption and/or ingestion within the gastrointestinal tract, and, in use, is swallowed and then consumed and/or ingested within that tract.

Lipid-based carrier systems within which solid particles of C21 or salt thereof are suspended may be in the form of solids at room temperature (fats) or, more preferably, may in the form of liquids at room temperature (oils). Particles of C21 or salt thereof may nevertheless be suspended in either form of lipid carrier.

Appropriate pharmaceutically-acceptable capsules include soft-shell or hard-shell capsules, which can be made from gelatin, cellulose polymers, e.g. hydroxypropyl methylcellulose (HPMC or hypromellose), hypromellose acetate succinate (HPMCAS), starch polymers, pullulan or other suitable materials, for example by way of standard capsule filling processes.

However, we prefer that the capsules are soft-shell, single-piece capsules, for example soft gelatin capsules, in which a single-piece gelatin capsule is filled with a lipid-based suspension of C21 or salt thereof, and thereafter sealed hermetically as a single piece, for example with a drop of gelatin solution. Gelatin may be obtained from any source (e.g. porcine and bovine sources), but it should be noted that there are vegan alternatives to soft gelatin capsules.

Soft gelatin capsule shells may comprise one or more plasticisers, such as xylitol, sorbitol, polyglycerol, non-crystallizing solutions of sorbitol, glucose, fructrose and glucose syrups, more preferably glycerin/glycerol, sorbitol and/or proprietary plasiticizers, such as Anidrisorbs (proprietary mixtures of sorbitol, sorbitans, maltitol and mannitol, Roquette Freres, including Anidrisorb 85/70 (a liquid sorbitol-mannitol-hydrolyzed starch plasticizer)). Soft gelatin capsule shells optionally comprise one or more flavouring agents, colouring agents and/or opacifiers (such as titanium dioxide).

Such capsules may be of any shape (e.g. oblong, round, oval, tubular, etc.) and of any size (e.g. 3 to 24 oblong, 1 to 20 round, 2 to 20 oval, 5 to 120 tube, etc.). Preferred capsule sizes will hold a volume of between about 0.3 and about 1.0 mL.

It is an essential aspect of the invention that C21 or pharmaceutically-acceptable salt thereof is suspended in a pharmaceutically-acceptable, hydrophobic, lipid-based carrier, and that, accordingly, the C21 or salt thereof is essentially insoluble within that carrier under normal storage conditions. By ‘essentially insoluble’ we include that C21 or salt thereof has a solubility within that carrier that is no more than about 0.015 mg of C21 or salt thereof per gram of carrier.

In this way, because of the carrier's dual properties of hydrophobicity and lack of propensity to dissolve C21 or salt thereof, the active ingredient is essentially not exposed to amounts of water that may catalyze degradation, for example as described hereinafter.

We have found, surprisingly, that there are relatively few lipid-based carrier materials that meet these requirements and are therefore able to stabilize C21 or salts thereof at ambient temperatures in dosage forms of the invention.

Hydrophobic lipid-based carrier materials in which C21 or salt thereof must be insoluble, as hereinbefore defined, may comprise a non-polar oil or fat that is essentially non-miscible with water. It is preferred that the lipid-based carrier is mainly comprised of triacylglycerols (also known as ‘triglycerides’), which are esters formed by reaction of all three hydroxyl groups of a glycerol moiety with fatty (carboxylic) acids.

Lipids may thus contain saturated or unsaturated chain fatty acids, which chain can range from 1 carbon atom up to 30 carbon atoms, including up to 26 carbon atoms, such as up to 22 carbon atoms, including 8, 10, 12, 14, 16, 18 or 20 carbon atoms, etc.

Saturated fatty acids that may be mentioned include acetic acid (2), propionic acid (3), butyric acid (4), valeric acid (5), caproic acid (6), enanthic acid (7), caprylic acid (8), pelargonic acid (9), capric acid (10), undecylic acid (11), lauric acid (12), tridecylic acid (13), myristic acid (14), pentadecylic acid (15), palmitic acid (16), margaric acid (17), stearic acid (18), nonadecylic acid (19), arachidic acid (20), heneicosylic acid (21), behenic acid (22), tricosylic acid (23), lignoceric acid (24), pentacosylic acid (25), cerotic acid (26), carboceric acid (27), montanic acid (28), nonacosylic acid (29) and melissic acid (30), wherein the numbers in brackets are the number of carbon atoms in the fatty acid molecule.

Unsaturated fatty acids that may be mentioned include crotonic acid (4:1), as well as ω-3 unsaturated fatty acids, such as octanoic acid (8:1), decanoic acid (10:1), decaclienoic acid (10:2), lauroleic acid (12:1), laurolinoleic acid (12:2), myristovaccenic acid (14:1), rnyristolinoleic acid (14:2), myristolinolenic acid (14:3), palmitolinolenic acid (16:3), hexadecatrienoic acid (16:3), palmitidonic acid (16:4), α-linolenic acid (18:3), stearidonic acid (18:4), 11,14,17-eicosatrienoic acid (20:3), eicosatetraenoic acid (20:4), eicosapentaenoic acid (20:5), heneicosapentaenoic acid (21:5), clupanodonic acid (22:5), docosahexaenoic acid (22:6), 9,12,15,18,21-acid (24:5), herring acid (24:6) and 6,9,12,15,18,21-tetracosahexaenoic acid (24:6); ω-5 unsaturated fatty acids, such as myristoleic acid (14:1), palmitovaccenic acid (16:1), α-eleostearic acid (18:3), β-eleostearic acid (trans-18:3), punicic acid (18:3), 7,10,13-octadecatrienoic acid (18:3), 9,12,15-eicosatrienoic acid (20:3) and β-eicosatetraenoic acid (20:4); ω-6 unsaturated fatty acids, such as tetradecenoic acid (14:1), 12-octadecenoic acid (18:1), linoleic acid (18:2), linolelaidic acid (trans-18:2), γ-linolenic acid (18:3), calenclic acid (18:3), pinolenic acid (13:3), 11,14-eicosadienoic acid (20:2); dihomo-linoleic acid (20:2), dihomo-γ-linolenic acid (20:3), arachidonic acid (20:4), docosadienoic acid (22:2), adrenic acid (22:4), osbond acid (22:5), tetracosatetraenoic acid (24:4) and tetracosapentaenoic acid (24:5); ω-7 unsaturated fatty acids, such as 5-dodecenoic acid (12:1), 7-tetradecenoic acid (14:1), palmitoleic acid (16:1), vaccenic acid (18:1), rumenic acid (18:2), paullinic acid (20:1), 7,10,13-eicosatrienoic acid (20:3), 15-docosenoic acid (22:1) and 17-tetracosenoic acid (24:1); ω-9 unsaturated fatty acids, such as hypogeic acid (16:1), oleic acid (13:1), elaidic acid (trans-18:1), gondoic acid (20:1), 8,11-eicosadienoic acid (20:2), erucic acid (22:1), nervonic acid (24:1), mead acid (20:3) and ximenic acid (26:1); ω-10 unsaturated fatty acids, such as sapienic acid (16:1); ω-11 unsaturated fatty acids, such as gadoleic acid (20:1); and ω-12 unsaturated fatty acids, such as 4-hexadecenoic acid (16:1), petroselinic acid (18:1) and eicosenoic acid (20:1), wherein the numbers in brackets are, respectively, the number of carbon atoms, and number of unsaturated (i.e. double) bonds, in the fatty acid molecule.

Fatty acids that may be mentioned include caproic acid, caprylic acid, capric acid, lauric acid, myristic acid, palmitic acid, stearic acid, oleic acid, ricinoleic acid, linoleic acid, linolenic acid, eicosenoic acid, behenic acid and erucic acid.

Triglycerides may be naturally-occurring oils or fats, may be semi-synthetic or may be synthetic.

Naturally-occurring oils or fats may be obtained from an animal or, more preferably, vegetable sources, such as seeds, kernels, or fruits.

Naturally-occurring vegetable oils comprise, principally, triglycerides, which are mixtures of glycerides with differing fatty acid chain lengths.

Naturally-occurring pharmaceutically-acceptable oils that fall into this category include sunflower oil, soybean oil, corn oil, grape seed oil, rapeseed oil, sesame oil, almond oil, apricot kernel oil, cotton seed oil, palm kernel oil, castor oil, olive oil, palm oil and coconut oil (for respective compositions see, for example, Occurrence and Characteristics of Oils and Fats at pages 47-224 in Padley, Gunstone and Harwood (Eds.), The Lipid Handbook., Chapman & Hall, London, 1994).

When employed in dosage forms of the invention, naturally-occurring oils should be pharmaceutical grade and should therefore preferably be refined after extraction from their natural source(s). This may be done using techniques that are well known to those skilled in the art.

Preferred oils include one or more of sesame oil, corn oil, palm kernel oil, coconut oil or soya oil.

Semi-synthetic and synthetic lipid-based carrier systems may be made using techniques that are well known to those skilled in the art, for example separation, interesterification, fat splitting and transesterification (glycerolysis).

Semi-synthetic and synthetic lipid based carrier systems thus include those that are typically in the form of oils, including short chain (C₁ to C₅) triglycerides (such as triacetin) and medium chain (C₆ to C₁₂) triglycerides (the primary component of the naturally-occurring oils palm kernel and coconut oils, such as capric triglycerides, more specifically Miglyol 812N); and those that are often in the form of semi-solid fats, including long chain (C₁₄ to C₂₂) triglycerides (such as Gelicure 43/10).

Whatever form of hydrophobic lipid-based carrier system is employed, it is preferred that the principal component of the carrier system comprises at least about 85% triacylglycerols, more preferably at least about 90% triacylglycerols, and especially at least about 95% triacylglycerols.

Mixtures of any of the above-mentioned naturally-occurring, semi-synthetic and/or synthetic lipid-based carrier materials may be employed.

Compositions of the dosage forms of the invention comprising C21 or salt thereof suspended in a lipid-based carrier as hereinbefore defined may, once prepared, be thereafter loaded into capsules. In view of the fact that it is preferred that such compositions are prepared in an essentially water-free state, such loading also preferably takes place in a manner in which it is kept in such a state.

By ‘essentially water free’, we include that appropriate precautions are taken to ensure that both particles of C21 or salt thereof, and the essential excipients in which it is suspended, are individually prepared and/or provided in a manner in which they are essentially dry, and are also mixed together to form dry mixture in an environment in which they are kept essentially dry.

By ‘essentially dry’ or ‘essentially free of water’, we include that the composition comprising C21/salt and essential excipients comprises, as a whole, no more that about 5%, including no more than about 2%, such as no more than about 1%, including no more than about 0.5%, such as about 0.1% water or less.

In this respect, although pharmaceutically-acceptable capsule materials may contain residual amounts of water, in accordance with the invention, the presence of the lipid-based carrier material with the properties as hereinbefore defined means that ingress of water into the composition from the capsule material is minimised, so protecting the highly sensitive C21 or salt thereof from contact with water and therefore, in the presence of light, degradation.

It is nevertheless preferred (although not necessarily essential) to package dosage forms of the invention in a manner that keeps the dosage form itself dry and protected from light. This may include hermetically-sealed packaging, use of deliquescent materials, etc.

C21 or salt thereof is presented in the form of particles, which may be amorphous or crystalline or a mixture of the two. Preferred particles are of a size that will not lead to sedimentation, either during formation of the suspension, the capsule loading process, or upon storage.

In this respect, C21 or salt thereof may be provided for suspension in the lipid-based carrier in the form of a plurality of primary (i.e. non-agglomerated) particles typically having a weight- and/or a volume-based mean diameter of no more than about 1,000 μm, such as about 500 μm, including about 250 μm, preferably no more than about 100 μm, including no more than about 50 μm, such as about 20 μm, or no more than about 10 μm. Although there is no lower limit on particle sizes that may be employed in the suspension, for ease of manufacture, we prefer that primary particles of C21 or salt thereof have weight- and/or volume-based mean diameter of no less than about 1 μm, such as about 2 μm, including about 3 μm.

As used herein, the term ‘weight based mean diameter’ will be understood by the skilled person to include that the average particle size is characterised and defined from a particle size distribution by weight, i.e. a distribution where the existing fraction (relative amount) in each size class is defined as the weight fraction, as obtained by e.g. sieving (e.g. wet sieving). The term ‘volume based mean diameter’ is similar in its meaning to weight based mean diameter, but will be understood by the skilled person to include that the average particle size is characterised and defined from a particle size distribution by volume, i.e. a distribution where the existing fraction (relative amount) in each size class is defined as the volume fraction, as measured by e.g. laser diffraction. Particle sizes may also be measured by standard equipment, such as a dry particle size measurement technique, including dry dispersion technologies available from manufacturers such as Sympatec GmbH (Clausthal-Zellerfeld, Germany). Other instruments that are well known in the field may be employed to measure particle size, such as equipment sold by e.g. Malvern Instruments, Ltd. (Worcestershire, UK), Shimadzu (Kyoto, Japan) and (Elzone, Micromeritics (USA; electrical sensing zone method).

By particles having weight- and/or volume-based mean diameters within the above limits, we include mean diameters of particles when prepared and/or prior to suspension in the lipid-based carrier, when so suspended and/or prior to being loaded into capsules. It will be appreciated that some aggregation of primary particles to form secondary particles may occur during handling and/or processing of active ingredient. This should nevertheless be minimised.

Primary particles of C21 or salt thereof may be prepared by an appropriate technique, such as precipitation, cutting (e.g. by way of dissolution in a supercritical fluid under pressure, followed by rapid expansion), spray drying, or may, if appropriate, be micronized by techniques that are well known to those skilled in the art, such as grinding, dry milling, jet milling, wet milling and/or crushing.

Particles may also be sieved to separate into a desired size fraction, and/or screened to break up agglomerates and/or remove fine material. In either case, unused undersized (fine), and oversized, material may be reworked to avoid waste. Alternatively, particles may be separated into appropriate particle sizes using cyclonic separation, by way of an air classifier, sedimentation, force-field fractionation and/or elutriation.

It is very important to ensure that, prior to loading of the suspension into capsules, it comprises C21 or salt thereof homogenously and evenly distributed throughout the suspension, to ensure close homogeneity of active ingredient following such loading into capsules.

In this respect, C21 or salt thereof is preferably provided in the form of particles with a relative narrow particle size distribution (PSD), as measured by standard techniques and art-accepted parameters, including mass median diameter (D₅₀; the log-normal mass median diameter), the average particle size by mass and/or the diameter at which 50% of the mass in the cumulative PSD are contained) and/or geometric standard deviation (GSD or σ_{g} as measured by the formula D_{84.13}/D₅₀ or D₅₀/D_{15.78}, where D_{84.13} and D_{15.78} are respectively the diameters at which 84.13% and 15.78% of the mass are contained, and D₅₀ is as hereinbefore defined). Such parameters may be measured and calculated in-process using any appropriate sampling method and particle size measurement technique as described hereinbefore.

It is preferred in this respect that C21 or salt thereof has a PSD with a GSD that is less than about 4, such as less than about 3.

Although C21 or salt thereof may be selected and/or provided with such a PSD and/or GSD using one or more of the above techniques to provide a stable suspension with an even distribution of C21/salt particles within that suspension, it is important to ensure thorough mixing of C21/salt with the lipid-based carrier system to ensure that an even distribution of active ingredient particles within the carrier is provided prior to loading. This is particularly so in the case of a bulk suspension that is employed as part of a capsule-loading process, where it is important to ensure that the mixture is homogeneous, not only at the outset, but also that this homogeneity is retained during the loading process to ensure dose homogeneity within a production batch.

The terms ‘homogeneous’ and ‘distributed homogeneously’ in the context of the invention mean that there is a substantially uniform content of C21 or salt thereof throughout the lipid-based carrier material. In other words, if multiple (e.g. at least, 2, more preferably about 6, such as about 10 up to about 30 or more if needed) samples are taken from a suspension in accordance with the invention, the measured content of active ingredient that is present as between such samples gives rise to a standard deviation from the mean amount (i.e. the coefficient of variation and/or relative standard deviation) of less than about 8%, such as less than about 6%, for example less than about 5%, particularly less than about 4%, e.g. less than about 3% and preferably less than about 2%.

Thus, in accordance with the invention, C21 or pharmaceutically-acceptable salt thereof may be made and stored in the form of a composition that may be directly loaded into capsules to make a dosage form of the invention, and furthermore, once made, dosage forms of the invention may be stored under normal storage conditions, with an insignificant degree of changes in physico-chemical properties.

If the lipid-based carrier system is in the form of a fat (i.e. a solid or a semi-solid at or around normal manufacturing temperatures and/or product storage temperatures), the skilled person will appreciate that the fat will need to be melted by raising the temperature prior to mixing.

Further, in order to ensure that such a suspension provides for a stable, homogeneous, even distribution of active ingredient within the carrier, if necessary, the lipid-based carrier system (and particularly those that are in the form of a liquid oil at or around normal manufacturing temperatures and/or product storage temperatures) may further comprise a thickening agent to avoid particle aggregation and/or sedimentation, such as microcrystalline cellulose and carboxymethylcellulose sodium, as well as blends of mono, di- and triglycerides with PEG esters of unsaturated fats, such as Gelucire 43/01, hydrogenated vegetable oil, beeswax, paraffin wax, etc.

By presenting C21, or salt thereof, in the form of a suspension of particles in accordance with the invention, we have found that dosage forms of the invention are not only capable of delivering a consistent and/or uniform dose of active ingredient, but also that it is possible to ensure that the active ingredient remains in a form in which it is both physically and chemically stable during and/or after manufacture, under normal storage conditions, and/or during use.

C21, or pharmaceutically-acceptable salt thereof, can be made and stored in the form of a suspension composition that is to be loaded into capsules to make a dosage form of the invention, but also that, once made, dosage forms of the invention may be stored under normal storage conditions, with an insignificant degree of changes in physico-chemical properties of the dosage form, suspension composition contained therein and/or, most importantly, active ingredient, over time.

An ‘insignificant degree of changes in physico-chemical properties’ thus includes that suspensions comprising C21/salt in a lipid-based carrier as hereinbefore described, before having been loaded into capsules and after (i.e. in the form of a dosage form of the invention), possess both physical stability and chemical stability.

By ‘chemical stability’, we include that suspensions comprising C21/salt in lipid-based carriers, and dosage forms of the invention, may be stored (with or without appropriate pharmaceutical packaging), under normal storage conditions, with an insignificant degree of chemical degradation or decomposition of the dosage forms of the invention, suspensions contained therein and, particularly, the active ingredient.

By ‘physical stability’, we include that suspensions comprising C21/salt in lipid-based carriers, and dosage forms of the invention, may be stored (with or without appropriate pharmaceutical packaging), under normal storage conditions, with an insignificant degree of physical transformation, such as aggregation or sedimentation as described above, or changes in the nature and/or integrity of the dosage forms of the invention, suspensions contained therein and, particularly, the active ingredient, including dissolution, solvatization, solid state phase transition, etc.

Examples of ‘normal storage conditions’ include temperatures of between minus 80 and plus 50° C. (preferably between 0 and 40° C. and more preferably ambient temperature, such as between 15 and 30° C.), pressures of between 0.1 and 2 bars (preferably atmospheric pressure), relative humidities of between 5 and 95% (preferably 10 to 60%), and/or exposure to 460 lux of UV/visible light, for prolonged periods (i.e. greater than or equal to six months).

Under such conditions, C21, a salt thereof, and/or lipid-based compositions containing them, may be found to be less than about 15%, more preferably less than about 10%, and especially less than about 5%, physically and/or chemically transformed as hereinbefore defined. The skilled person will appreciate that the above-mentioned upper and lower limits for temperature and pressure represent extremes of normal storage conditions, and that certain combinations of these extremes will not be experienced during normal storage (e.g. a temperature of 50° C. and a pressure of 0.1 bar).

Dosage forms of the invention may include other excipients that are well known to those skilled in the art for peroral delivery of active ingredients. For example, dosage forms of the invention may also impart, or may be modified to impart, an immediate, or a modified, release of active ingredient(s).

Additional excipients may be commercially-available or otherwise are described in the literature, for example, Remington The Science and Practice of Pharmacy, 21st ed., Lippincott Williams and Wilkins, Philadelphia (2006) and the documents referred to therein, the relevant disclosures in all of which documents are hereby incorporated by reference. Otherwise, the preparation of suitable peroral formulations may be achieved non-inventively by the skilled person using routine techniques.

According to a further aspect of the invention there is provided a process for the production of a dosage form of the invention, which process comprises:
(a) mixing particles of C21 or a pharmaceutically-acceptable salt thereof with a pharmaceutically-acceptable, hydrophobic, lipid-based carrier in which C21 or salt thereof is essentially insoluble, to form a suspension of C21 or salt thereof in said lipid-based carrier; and
(b) loading said suspension from step (a) into a capsule that is suitable for peroral administration.

Pharmaceutically-acceptable salts of C21 include acid addition salts. Such salts may be formed by conventional means, for example by reaction of C21 in the form of the free acid (hereinafter ‘free C21’) with one or more equivalents of an appropriate acid, optionally in a solvent, or in a medium in which the salt is insoluble, followed by removal of said solvent, or said medium, using standard techniques (e.g. in vacuo, by freeze-drying or by filtration). Salts may also be prepared by exchanging a counter-ion of an active ingredient in the form of a salt with another counter-ion, for example using a suitable ion exchange resin. Preferred salts of C21 include HCl salts, alkaline earth salts, such as magnesium and calcium salts, and alkali metal salts, such as potassium or, preferably, sodium salts.

The amount of C21 or salt thereof in a dosage form of the invention will depend, and/or may be selected depending, upon the severity of the condition, or the expectation of such severity, as well as on the patient, to be treated, but may be determined by the skilled person. The mode of administration may also be determined by the timing and frequency of administration, as well as the severity of the condition.

Suitable lower daily doses of C21 in adult patients (average weight e.g. 70 kg), may be about 10 mg, such as about 20 mg, for example about 25 mg, per day. Suitable upper limits of daily dose ranges of C21 may be about up to about 900 mg, such as 600 nig, including about 400 mg and about 200 mg, such as about 100 nig, and including about 50 mg.

All of the above doses are calculated as the free C21. Doses may be split into multiple individual doses per day. Doses may be given between once and six, such as four times daily, preferably three times daily and more preferably twice daily.

In any event, the medical practitioner, or other skilled person, will be able to determine routinely the actual dosage, which will be most suitable for an individual patient, depending on the severity of the condition and route of administration. The above-mentioned dosages are exemplary of the average case; there can, of course, be individual instances where higher or lower dosage ranges are merited, and such are within the scope of this invention.

The dose administered to a patient, in the context of the present invention should be sufficient to effect an appropriate response in the patient over a reasonable timeframe (as described hereinbefore). One skilled in the art will recognize that the selection of the exact dose and composition and the most appropriate delivery regimen will also be influenced by inter alia the pharmacological properties of the formulation, the nature, stage and/or severity of the condition being treated, the physical condition and mental acuity of the recipient, including the age, condition, body weight, sex and response of the patient to be treated, and the stage/severity of the disease, and genetic differences between patients.

Dosage forms of the invention are useful in conditions where AT2 receptors are expressed and their stimulation is desired or required.

In this respect, dosage forms of the invention are indicated in the treatment of conditions characterised by vasoconstriction, fibrosis, inflammation, increased cell growth and/or differentiation, increased cardiac contractility, increased cardiovascular hypertrophy, and/or increased fluid and electrolyte retention, as well as skin disorders and musculoskeletal disorders.

Dosage forms of the invention are particularly indicated in the treatment and/or prevention of ILDs, such as sarcoidosis or fibrosis, more specifically PF and particularly IPF, as well as conditions that may trigger ILDs, such as systemic sclerosis, rheumatoid arthritis, myositis or systemic lupus erythematosus, or are otherwise associated with ILDs, such as pulmonary hypertension and/or pulmonary arterial hypertension.

Dosage forms of the invention may also exhibit thromboxane receptor activity. In this respect, dosage forms of the invention may have an inhibitory effect on platelet activation and/or aggregation (and thus e.g. an antithrombotic effect), and/or may reduce vasoconstriction and/or bronchoconstriction in a therapeutic manner.

Dosage forms of the invention are further indicated in the treatment of stress-related disorders, and/or in the improvement of microcirculation and/or mucosa-protective mechanisms.

Thus, dosage forms of the invention are expected to be useful in the treatment of disorders, which may be characterised as indicated above, and which are of, for example, the gastrointestinal tract, the cardiovascular system, the respiratory tract, the kidneys, the immune system, the eyes, the female reproductive (ovulation) system and the central nervous system (CNS).

Disorders of the gastrointestinal tract that may be mentioned include oesophagitis, Barrett's oesophagus, gastric ulcers, duodenal ulcers, dyspepsia (including non-ulcer dyspepsia), gastro-oesophageal reflux, irritable bowel syndrome (IBS), inflammatory bowel disease (IBD), pancreatitis, hepatic disorders (such as hepatitis), gall bladder disease, multiple organ failure (MOF) and sepsis. Other gastrointestinal disorders that may be mentioned include xerostomia, gastritis, gastroparesis, hyperacidity, disorders of the bilary tract, coelicia, Crohn's disease, ulcerative colitis, diarrhoea, constipation, colic, dysphagia, vomiting, nausea, indigestion and Sjögren's syndrome.

Disorders of the respiratory tract that may be mentioned include inflammatory disorders, such as asthma, obstructive lung diseases (such as chronic obstructive lung disease), pneumonitis, pulmonary hypertension, and adult respiratory distress syndrome.

Disorders of the kidneys that may be mentioned include renal failure, diabetic nephropathy, nephritis and renal hypertension.

Disorders of the eyes that may be mentioned include diabetic retinopathy, premature retinopathy and retinal microvascularisation.

Disorders of the female reproductive system that may be mentioned include ovulatory dysfunction and endometriosis.

Cardiovascular disorders that may be mentioned include hypertension, cardiac hypertrophy, cardiac failure (including heart failure with preserved ejection fraction), artherosclerosis, arterial thrombosis, venous thrombosis, endothelial dysfunction, endothelial lesions, post-balloon dilatation stenosis, angiogenesis, diabetic complications, microvascular dysfunction, angina, cardiac arrhythmias, claudicatio intermittens, preeclampsia, myocardial infarction, reinfarction, ischaemic lesions, erectile dysfunction and neointima proliferation.

Disorders of the CNS that may be mentioned include cognitive dysfunctions, dysfunctions of food intake (hunger/satiety) and thirst, stroke, cerebral bleeding, cerebral embolus and cerebral infarction, multiple sclerosis (MS), Alzheimer's disease and Parkinson's disease.

Dosage forms of the invention may also be useful in the modulation of growth metabolism and proliferation, for example in the treatment of ageing, hypertrophic disorders, prostate hyperplasia, autoimmune disorders (e.g. arthritis, such as rheumatoid arthritis, or systemic lupus erythematosus), psoriasis, obesity, neuronal regeneration, the healing of ulcers, inhibition of adipose tissue hyperplasia, stem cell differentiation and proliferation, fibrotic disorders, cancer (e.g. in, or of, the gastrointestinal tract (including the oesophagus or the stomach), the prostate, the breast, the liver, the kidneys, as well as lymphatic cancer, lung cancer, ovarian cancer, pancreatic cancer, hematologic malignancies, etc.), apoptosis, tumours (generally) and hypertrophy, diabetes, neuronal lesions and organ rejection.

Dosage forms of the invention are also useful in the treatment of stroke, spinal cord injury, sickle cell disease, muscular dystrophy, cancer treatment-related cardiotoxicity, peripheral neuropathy and, in particular, systemic sclerosis.

In addition, dosage forms of the invention may be useful in the treatment of respiratory virus-induced tissue damage, which damage may include injury and/or dysfunction of relevant tissues. Relevant tissues include (e.g. mucosal) tissues of the respiratory tract, and especially those of the lung. Relevant tissue thus includes the respiratory epithelium, which moistens the airways and protects against invasion of pathogens such as viruses.

Respiratory viruses that may be mentioned in this respect include influenza viruses, such as influenza A virus (e.g. H1N1 and H3N2 viruses), influenza B virus or influenza C virus), and, more particularly, coronaviruses, including severe acute respiratory syndrome (SARS) coronaviruses, such as SARS coronavirus (SARS-CoV) and, particularly, the novel SARS coronavirus 2 (SARS-CoV-2, previously known as ‘2019-nCoV’ or ‘novel coronavirus 2019’), which is the virus that causes coronavirus disease 2019 (COVID-19), of which there are many genetic variants.

By ‘treatment of tissue damage’, we include that C21 and salts thereof may not only have a beneficial effect on tissue damage in the respiratory tract that has been caused by such a virus, but that it may also prevent and/or mitigate the damage that would otherwise have been caused by that virus in the respiratory tract, which occurs when the relevant virus enters e.g. epithelial cells in the respiratory tract.

Thus, C21 and salts thereof may abrogate or prevent the development of diseases that are caused by such virally-induced tissue damage and/or the symptoms of such damage or diseases.

In this respect, C21 and salts thereof may treat, and/or arrest the progress of, diseases that are being, or have been, caused by respiratory viruses (i.e. diseases such as influenza, as well as acute lung injury acute lung injury (ALI), acute respiratory distress syndrome (ARDS), particularly SARS and, more particularly, COVID-19) and their sequelae. C21 and salts thereof may also treat and/or prevent the damage that is being, or has been, caused by such viruses, which includes treating and/or preventing the symptoms of such respiratory diseases, which symptoms include cough, dyspnea, respiratory distress (as manifest by e.g. the need for supplementary/supplemental oxygen (which may be administered by a face mask or via nasal cannula (high flow or otherwise)), and/or mechanical ventilation/extra-corporeal membrane oxygenation), respiratory failure, and/or pneumonia, which may occur directly (viral pneumonia) and/or indirectly (bacterial pneumonia resulting from secondary bacterial infections, which is common in influenza), as well as subsequent fibrosis resulting from inflammation in the lungs and other organs (e.g. the heart and kidneys). Further, C21 and salts thereof may prevent or arrest the progress of respiratory virus-induced morbidity and/or mortality, and C21 may treat, and/or arrest the development of any of the chronic symptoms identified above.

In addition, dosage forms of the invention may also be useful in the treatment or prevention of any fibrotic condition of one or more internal organs characterised by the excessive accumulation of fibrous connective tissue, and/or in the treatment or prevention of fibrogenesis and the morbidity and mortality that may be associated therewith. Such fibrosis may be associated with an acute inflammatory condition, such as acute respiratory distress syndrome (ARDS), SARS, and multiple-organ inflammation, injury and/or failure, which may be caused by internal or external trauma (e.g. injury), or by an infection.

Such conditions may thus result from sepsis or septic shock caused by a viral, bacterial or fungal infection. Furthermore, acute lung injury, ARDS and, particularly, SARS may be caused by viruses, such as coronaviruses, include SARS-CoV-2, which may result in internal tissue damage and/or dysfunction of relevant internal (e.g. mucosal) tissues, and/or the cells that comprise them, such as the respiratory epithelium. Such tissue damage may in turn give rise to severe fibrosis. For example, the SARS disease caused by SARS-CoV-2 (coronavirus disease 2019 or COVID-19) is known in many cases to result in fibrosis.

However, dosage forms of the invention are also especially useful in the treatment or prevention of ILDs as defined herein, including sarcoidosis or fibrosis, more specifically pulmonary fibrosis and particularly IPF, as well as conditions that may trigger ILDs, such as systemic sclerosis, rheumatoid arthritis, myositis or systemic lupus erythematosus, or are otherwise associated with ILDs, such as pulmonary hypertension and/or pulmonary arterial hypertension.

The term ‘ILD’ will be understood by those skilled in the art to include any pulmonary condition characterized by an abnormal healing response, including chronic inflammation, reduced lung function and/or scarring, irrespective of the cause, such as sarcoidosis, PF and, especially, IPF. The term may also include diseases and/or conditions that are known to lead to, and/or be causes of, such pulmonary conditions, such as systemic sclerosis. In this respect there is further provided a dosage form of the invention for use in the condition that leads to and/or is a cause of an ILD, such as PF or IPF, including systemic sclerosis.

In the treatment of PF, including IPF, dosage forms of the invention may have an anti-fibrotic effect, with reduction of fibrosis and prevention of further deposition of extra cellular matrix. Dosage forms of the invention may affect lune scarring/wound healing and also have an anti-apoptotic effect, thereby preventing apoptosis for alveolar endothelial cells, being an initiating factor for the development of PF. Dosage forms of the invention may also have an anti-proliferative effect, thus reducing the cancer-like proliferation of fibroblasts and myofibroblasts in PF. Dosage forms of the invention may also improve vascular remodelling in PF, thereby reducing secondary pulmonary hypertension. Finally, dosage forms of the invention may demonstrate anti-inflammatory and anti-cytokine effects.

According to a further aspect of the present invention, there is provided a method of treatment of any of the aforementioned conditions, including respiratory viral damage and, more particularly, an ILD, including PF, and in particular IPF, which method comprises administration of a therapeutically effective amount of a dosage form of the invention to a person suffering from, or susceptible to, such a condition.

According to a yet further aspect of the present invention, there is provided a method of treatment of respiratory virus-induced tissue damage in a subject, which method comprises administration of a therapeutically effective amount of a dosage form of the invention to a subject in need of such treatment, particularly in which:
the tissue that is damaged is lung tissue, including the respiratory epithelium;
the damage comprises injury and/or dysfunction of the mucosal tissue of the respiratory tract caused by a respiratory virus;
the treatment includes treatment, and/or arresting the progress, of a disease that is being, or has been, caused by the virus;
the respiratory virus is a coronavirus, such as SARS-Coll-2, and the disease is a SARS, such as COVID-19; or the respiratory virus is an influenza virus, and the disease is influenza;
the treatment includes treatment of the symptoms of the disease that is being, or has been, caused by the relevant virus;
the symptoms of the damage or the disease include one or more of cough, dyspnea, respiratory distress (which may be manifest by the need for supplementary oxygen and/or mechanical ventilation), respiratory failure, pneumonia, fibrosis in one or more internal organs, including the lungs, the heart and/or the kidneys; and/or
the treatment includes prevention of respiratory virus-induced morbidity and/or mortality in one or more of the foregoing conditions.

The dosage forms of the invention are indicated both in the therapeutic, palliative, and/or diagnostic treatment (e.g. during diagnostic workup if a condition is suspected), as well as the prophylactic treatment (by which we include preventing and/or abrogating deterioration and/or worsening of a condition) of any of the above conditions.

‘Patients’ include avian and mammalian (particularly human) patients. Human patients include both adult patients as well as pediatric patients, the latter including patients up to about 24 months of age, patients between about 2 to about 12 years of age, and patients between about 12 to about 16 years of age. Patients older than about 16 years of age may be considered adults for purposes of the present invention. These different patient populations may be given different doses of C21 or salt thereof.

It is preferred, in the treatment of certain conditions such as respiratory virus-induced tissue damage, that C21 or a pharmaceutically-acceptable salt thereof is administered to adult patients, more particularly subjects that are over the age of about 20, such as over the age of about 30, including over the age of about 40, more preferably over the age of about 50, especially over the age of about 60, particularly over the age of about 70, and more particularly over the age of about 80 years of age; and/or to patients (whether or not such patients are in one of the age groups specified above) with one or more of the following underlying medical conditions:
chronic (long-term) respiratory diseases, such as pulmonary fibrosis, pulmonary hypertension, pulmonary arterial hypertension, other ILDs, asthma, chronic obstructive pulmonary disease (COPD), emphysema or bronchitis
chronic cardiovascular (e.g. heart) disease, such as heart failure, atrial fibrillation or hypertension
chronic kidney disease
chronic liver disease, such as hepatitis
chronic neurological conditions, such as Parkinson's disease, motor neurone disease, multiple sclerosis, a learning disability or cerebral palsy
diabetes
problems with a patient's spleen—for example, sickle cell disease or if the spleen has been removed
a weakened immune system as the result of conditions, such as HIV and AIDS, or medicines such as steroid tablets or chemotherapy
obesity (e.g. a body mass index (BMI) of 40 or above)
pregnancy.

In this respect, according to several further aspects of the invention there is provided a method of treatment and/or prevention of one or more the following conditions:
post-acute sequelae of e.g. SARS-CoV-2 infection (PASC), such as what is known as ‘long COVID’, ‘chronic COVID syndrome’ (CCS) and/or ‘long-haul COVID’;
acute kidney injury and/or chronic kidney disease;
respiratory diseases such as pulmonary fibrosis, pulmonary hypertension, pulmonary arterial hypertension, asthma, chronic obstructive pulmonary disease (COPD), emphysema and/or bronchitis; and
cardiovascular diseases such as myocardial infarction, heart failure, atrial fibrillation, hypertension or thrombosis and/or embolization in e.g. the heart, lungs and/or brain,

all of which may be induced, directly or indirectly, by respiratory viruses (such as SARS-CoV-2), which method comprises administering C21 or a pharmaceutically-acceptable salt thereof to a subject in need of such treatment and/or prevention.

In relation to (for example) acute treatment of respiratory virus-induced tissue damage, doses of C21 or salt thereof may be administered between once and four times (e.g. between 1 and 3 times) daily for up to three (e.g. two) months, such as one month, including up to three weeks, e.g. up to one week, such as 4 days or 3 days. Such treatment periods may be repeated as appropriate.

In the case of the development of one or more of the chronic symptoms identified hereinbefore, such as fibrosis of the lungs and other internal organs, treatment with C21 or salt thereof may, in addition to and/or instead of the above-mentioned acute dosing regimens, be continuous and/or as needed/required.

Relevant active ingredients that may be used in combination therapy with C21 in the treatment of patients with viral infections include more the variously-applied standard treatments for viral infections, including antibody therapies (e.g. LY-CoV555/LY-CoV016 (bamlanivimab and etesevimab), LY-CoV555 (bamlanivimab, Eli Lilly), REGN-COV2 (casirivimab and imdevimab), REGN3048-3051, TZLS-501, SNG001 (Synairgen), eculizumab (Soliris; Alexion Pharmaceuticals), ravulizumab (Ultomiris; Alexion Pharmaceuticals), lenzilumab, leronlimab, tocilizumab (Actemra; Roche), sarilumab (Kevzara; Regeneron Pharma), and Octagam (Octapharma)), antiviral medicines (e.g. oseltamivir, remdesivir, favilavir, molnupiravir, simeprevir, daclatasvir, sofosbuvir, ribavirin, umifenovir, lopinavir, ritonavir, lopinavir/ritonavir (Kaletra; AbbVie Deutschland GmbH Co. KG), teicoplanin, baricitinib (Olumiant; Eli Lilly), ruxolitinib (Jakavi; Novartis), tofacitinib (Xeljanz; Pfizer), the TMPRSS2 inhibitor, camostat, or camostat rnesylate, Actembra (Roche), TZLS-501, AT-100 (rhSP-D), MK-7110 (CD24Fc; Merck)), OYA1 (OyaGen9), BPI-002 (BeyondSpring), NP-120 (Ifenprodil; Algernon Pharmaceuticals), Galidesivir (Biocryst Pharma), antiinflammatory agents (e.g. NSAIDs, such as ibuprofen, ketorolac, naproxen, and the like), chloroquine, hydroxychloroquine, interferons (e.g. interferon beta (interferon beta-1a), tocilizumab (Actemra), lenalidomide, pornalidomide and thalidomide), analgesics (e.g. paracetamol or opioids), antitussive agents (e.g. dextrornethorphan), vaccinations (e.g. INO-4800 by Inovio Pharmaceuticals and Beijing Advaccine Biotechnology, if available), COVID-19 convalescent plasma (CCP) and/or passive antibody therapy with antibodies from blood of people who have recovered from infection with SARS-CoV or SARS-CoV-2.

Relevant active ingredients that may be used in combination therapy with C21 in the treatment of ILDs, such as IPF include, for example, anti-fibrotics (e.g. nintedanib and, particularly, pirfenidone); vitamins (e.g. vitamin B, C and D); mucolytics (e.g. acetylcysteine and ambroxol); corticosteroids, such as cortisone and prednisone; inflammation suppressants, such as cyclophosphamide; other immunosuppressants, such as azathioprine and mycophenolate mofetil; and antioxidants, such as N-acetylcysteine. Relevant active ingredients that may be used in combination therapy with C21 in the treatment of sarcoidosis include, for example, corticosteroicls, such as cortisone, prednisone and prednisolone; antimetabolites; immune system suppressants, such as methotrexate, azathioprine, leflunomide, mycophenoic acid/mycophenolate mofetil, cyclophosphamide; aminoquinolines; monoclonal anti-tumor necrosis factor antibodies, such as infliximab and adalimumab; immunomodulatory imide drugs, such as include lenalidomide, pomalidomide and, especially, thalidomide; the TNF inhibitor, etanercept; and painkillers, such as ibuprofen and paracetamol; cough suppressants and/or expectorants.

For the avoidance of doubt, ‘corticosteroids’ as mentioned above include both naturally-occurring corticosteroids and synthetic corticosteroids.

Naturally-occurring corticosteroids that may be mentioned include cortisol (hydrocortisone), aldosterone, corticosterone, cortisone, pregnenolone, progesterone, as well as naturally-occurring precursors and intermediates in corticosteroid biosynthesis, and other derivatives of naturally-occurring corticosteroids, such as 11-deoxycortisol, 21-deoxycortisol, 11-dehydrocorticosterone, 11-deoxycorticosterone, 18-hydroxy-11-deoxycorticosterone, 18-hydroxycorticosterone, 21-deoxycortisone, 11β-hydroxypregnenolone, 11β,17α,21-trihydroxypregnenolone, 17α,21-dihydroxypregnenolone, 17α-hydroxypregnenolone, 21-hydroxypregnenolone, 11-ketoprogesterone, 11β-hydroxyprogesterone, 17α-hydroxyprogesterone and 18-hydroxyprogesterone.

Synthetic corticosteroids that may be mentioned include those of the hydrocortisone-type (Group A), such as cortisone acetate, hydrocortisone aceponate, hydrocortisone acetate, hydrocortisone buteprate, hydrocortisone butyrate, hydrocortisone valerate, tixocortol and tixocortol pivalate, prednisolone, methylprednisolone, prednisone, chloroprednisone, cloprednol, difluprednate, fludrocortisone, fluocinolone, fluperolone, fluprednisolone, loteprednol, prednicarbate and triamcinolone; acetonides and related substances (Group B), such as amcinonide, budesonide, desonide, fluocinolone cetonide, fluocinonide, halcinonide, triamcinolone acetonide, ciclesonide, deflazacort, formocortal, fludroxycortide, flunisolide and fluocinolone acetonide, those of the (beta)methasone-type (Group C), such as beclomethasone, betamethasone, betamethasone clipropionate and betamethasone valerate, dexamethasone, fluocortolone, halometasone, mometasone and mometasone furoate, alclometasone and alclometasone dipropionate, clobetasol and clobetasol propionate, clobetasone and clobetasone butyrate, clocortolone, desoximetasone, diflorasone, difluocortolone, fluclorolone, flumetasone, fluocortin, fluprednidene and fluprednidene acetate, fluticasone, fluticasone furoate and fluticasone propionate, meprednisone, paramethasone, prednylidene, rimexolone and ulobetasol; those of the progesterone-type, such as flugestone, fluorometholone, medrysone and prebediolone acetate, and progesterone derivatives (progestins), such as chlormadinone acetate, cyproterone acetate, medrogestone, medroxyprogesterone acetate, megestrol acetate and segesterone acetate; as well as other corticosteroids, such as cortivazol and 6-methyl-11β,17β-dihydroxy-17α-(1-propynyl)androsta-1,4,6-trien-3-one.

Preferred corticosteroids include cortisone, prednisone, pralnisolone, methylprednisolone and, especially, dexamethasone.

Further, relevant active ingredients that may be used in combination therapy with C21 (e.g. to treat respiratory viral infections) include H2 receptor blockers, anticoagulants, anti-platelet drugs, as well as statins, antimicrobial agents and anti-allergic/anti-asthmatic drugs.

H2 receptor blockers that may be mentioned include famotidine. Anticoagulants that may be mentioned include heparin and low-molecular-weight heparins (e.g. bemiparin, nadroparin, reviparin, enoxaparin, parnaparin, certoparin, dalteparin, tinzaparin); directly acting oral anticoagulants (e.g. dabigatran, argatroban, rivaroxaban, apixaban, edoxaban, betrixaban, darexaban, otamixaban, letaxaban, eribaxaban, hirudin, lepirudin and bivalirudin); coumarin type vitamin K antagonists (e.g. coumarin, acenocoumarol, phenprocoumon, atromentin and phenindione) and synthetic pentasaccharide inhibitors of factor Xa (e.g. fondaparinux, idraparinux and idrabiotaparinux). Anti-platelet drugs that may be mentioned include irreversible cyclooxygenase inhibitors (e.g. aspirin and triflusal); adenosine diphosphate receptor inhibitors (e.g. cangrelor, clopidogrel, prasugrel, ticagrelor and ticlopidine); phosphocliesterase inhibitors (e.g. cilostazol); protease-activated receptor-1 antagonists (e.g. vorapaxar); glycoprotein IIB/IIIA inhibitors (e.g. abciximab, eptifibatide and tirofiban); adenosine reuptake inhibitors (e.g. dipyridamole); and thromboxane inhibitors (e.g. terutroban, ramatroban, seratrodast and picotarnide). Statins that may be mentioned include atorvastatin, simvastatin and rosuvastatin. Antimicrobial agents that may be mentioned include azithromycin, ceftriaxone, cefuroxime, doxycycline, fluconazole, piperacillin, tazobactam and teicoplanin. Anti-allergic/anti-asthmatic drugs that may be mentioned include chlorphenamine, levocetirizine and montelukast.

Further relevant active ingredients that may be used in combination therapy with C21 (e.g. to treat respiratory viral infections) include other AT2 agonists that are known in the art as well as in combination with AT1 receptor antagonists that are known in the art, and/or in combination with an inhibitor of angiotensin converting enzyme (ACE). Non-limiting but illustrative examples of AT1 receptor antagonists that can be used according to the embodiments include azilsartan, candesartan, eprosartan, fimasartan, irbesartan, losartan, milfasartan, olmesartan, pomisartan, pratosartan, ripiasartan, saprisartan, tasosartan, telmisartan, valsartan and/or combinations thereof. Non-limiting but illustrative examples of ACE inhibitors that can be used according to the embodiments include captopril, zofenopril, enalapril, ramipril, quinapril, perindopril, lisinopril, benazepril, imidapril, trandolapril, fosinopril, moexipril, cilazapril, spirapril, temocapril, alacepril, ceronapril, delepril, moveltipril, and/or combinations thereof.

Relevant patients may also (and/or may already) be receiving one or more of any of the treatments and/or other therapeutic agents mentioned above for the relevant condition based upon administration of one or more of such active ingredients, by which we mean receiving a prescribed dose of one or more of those active ingredients mentioned herein, prior to, in addition to, and/or following, treatment with C21 or a salt thereof.

Pharmaceutically-acceptable salts, and doses, of other active ingredients mentioned above include those that are known in the art and described for the drugs in question to in the medical literature, such as Martindale—The Complete Drug Reference, 38^{th} Edition, Pharmaceutical Press, London (2014) and the documents referred to therein, the relevant disclosures in all of which documents are hereby incorporated by reference.

Dosage forms of the invention have the advantage that they can be manufactured and stored under normal storage conditions, including without freezing and/or being exposed to light, maintaining pharmaceutically-acceptable physico-chemical stability of the composition contained with the capsule and, in particular, the active ingredient.

Dosage forms of the invention may also provide for an improved drug loading, enables high quantities/doses of active compound to be presented, and also efficient delivery of such higher doses in a consistent/uniform manner. This in turn enhances the effectiveness and efficiency of treatment and reduces costs for healthcare.

The uses/methods described herein may otherwise have the advantage that, in the treatment of one or more of the conditions mentioned hereinbefore, and in particulary ILDs and/or respiratory viral infections, they may be more convenient for the physician and/or patient than, be more efficacious than, be less toxic than, have a broader range of activity than, be more potent than, produce fewer side effects than, or that it may have other useful pharmacological properties over, similar methods (treatments) known in the prior art, whether used in those conditions or otherwise.

Wherever the word ‘about’ is employed herein, for example in the context of numbers or amounts, i.e. absolute amounts such as sizes (e.g. particle sizes), doses, weights or concentrations of (e.g. active) ingredients, ages, temperatures or time periods; or relative amounts including percentages and standard deviations, it will be appreciated that such variables are approximate and as such may vary by ±10%, for example ±5% and preferably ±2% (e.g. ±1%) from the actual numbers specified. In this respect, the term ‘about 10%’ means e.g. ±10% about the number 10, i.e. between 9% and 11%.

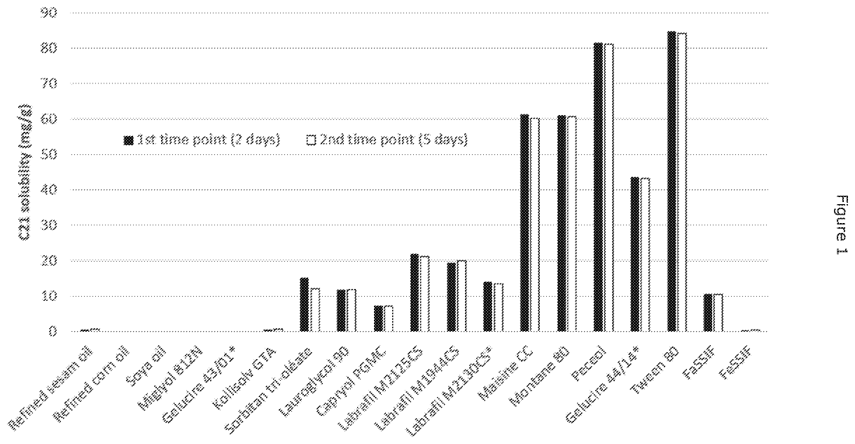

The invention is illustrated, but in no way limited, by the following examples, in which FIG. 1 shows solubility of C21 sodium salt in various lipid-based excipients.

### EXAMPLES

### Comparative Example 1

Solubility of C21 in Water

The solubility of free C21 was investigated in a number of different aqueous vehicles as summarised in Table 1 below.

Vehicles (with sources) were as follows: sodium chloride (Sigma), ethanol (99.5%, Kemetyl), polyethylene glycols (BASF), phosphate buffered saline (PBS) pH 7.4 (Sigma), buffer solution pH 2.00 (citric acid, sodium hydroxide, hydrogen chloride), buffer solution pH 4.00 (citric acid, sodium hydroxide), buffer solution pH 6.00 (citric acid, sodium hydroxide), buffer solution pH 8.00 (boric acid, sodium hydroxide, hydrogen chloride) and buffer solution pH 10.00 (boric acid, sodium hydroxide, hydrogen chloride) (all Merck), and purified water (Elga Option 4 water purifier).

Saturated solutions of free C21 (obtained from Syntagon AB, Södertälje, Sweden) were prepared in duplicates. The samples were kept magnetically stirred for 48 hours prior to analysis. For some samples, the added substance was dissolved and more was thereafter added to obtain saturated solutions.

After 48 hours, pH was measured and thereafter 1 mL of solution was withdrawn. Undissolved substance was removed by centrifugation (1500 rpm, 30 minutes). The supernatant was diluted 10 to 500 times with acetonitrile/H₂O, 30:70.

C21 content was measured by HPLC.

TABLE 1

| | Concentration | |
|---|---|---|
| Vehicle | (mg/ml)^{a} | pH |
| H₂O | 0.15 | 7.3 |
| 0.9% NaCl | 0.12 | 7.3 |
| 0.9% NaCl | 1.58^{b} | 8.3^{c} |
| 0.9% NaCl | 27.40 | 9.7^{c} |
| 0.9% NaCl/EtOH 95:5 v/v | 0.57^{b} | 7.9 |
| Buffer/Citric acid pH 2.0 | 3.95 | 2.3 |
| Buffer/Citric acid pH 4.0 | 0.08 | 4.0 |
| Buffer/Citric acid pH 6.0 | 0.06 | 6.0 |
| Buffer/PBS pH 7.4 | 0.24 | 7.7 |
| Buffer/Boric acid pH 8.0 | 0.50 | 7.9 |
| Buffer/Boric acid pH 10.0 | 19.10, 19.90 | 8.7 |
| PEG/H₂O (25:75) | 0.17 | 5.5 |
| PEG/H₂O (50:50) | 0.61 | 6.2 |
| PG/H₂O (10:90) | 0.22 | 7.5 |
| PG/H₂O (25:75) | 0.30 | 6.9 |
| PEG/EtOH/H₂O (40:10:50) | 0.83 | 6.1 |
| PG/EtOH/H₂O (40:10:50) | 0.79 | 6.3 |

^{a}Concentrations are mean values from two separate samples
^{b}Concentrations are mean values from two injections (one sample)
^{c}pH was adjusted by addition of NaOH

Above pHs of approximately 8.5, there is a marked increase in free C21 solubility, As much as 27.4 mg/mL is obtained at pH 9.7 in a 0.9% NaCl solution.

An increased solubility is also seen in the co-solvent systems studied. The change is however not as dramatic as by modification of pH.

The solubility of the sodium salt of C21 was measured by way of a similar experiment and was found to be considerably higher than free C21.

In this experiment, C21 sodium salt (Syntagon AB) was added to the vehicle, small amounts at a time. About 20-30 mg of the sodium salt was easily dissolved in all the vehicles tested. Salt was continuously added to the same sample in an attempt to obtain a saturated solution. In this way, higher amounts, such as 40-60 mg/mL could be dissolved. The solubility is probably even higher than this in the vehicles tested, but this was not established in view of the limited amount of drug compound available. The results are summarised in Table 2 below.

TABLE 2

| | Concentration | |
|---|---|---|
| Vehicle | (mg/mL)^{a} | pH |
| H₂O | >65 | 9.8 |
| 0.9% NaCl | >40 | 9.3 |
| PBS pH 7.4 | >40 | 9.4 |

^{a}Concentrations are mean values from two separate samples

### Comparative Example 2

Sensitivity of Aqueous Solutions of C21 to Light

The stability of free C21 in 0.9% NaCl pH 9.4 was investigated.

Solutions of 1 mg/mL of C21 were studied for four weeks under four different storage conditions. The solution was filtered through a 0.22 sterile syringe filter to minimize bacterial growth during the stability test. The samples were analysed by HPLC for purity.

The results are summarised in Table 3 below, in which the amount of C21 is given as a percentage of the initial amount of drug. Solution pHs were also measured and are shown within parenthesis in Table 3.

TABLE 3
Storage

| time | Amount of Free C21, % of initial | | | | | | | |
|---|---|---|---|---|---|---|---|---|
| (weeks) | 5° C., dark | | RT, dark | | RT, light | | 40° C., dark | |
| Initial^{a} | 100 | (9.4) | 100 | (9.4) | 100 | (9.4) | 100 | (9.4) |
| 1^{a} | 101 | (9.2) | 97 | (9.2) | 96 | (9.0) | 101 | (9.0) |
| 2^{b} | 107 | (9.2) | 109 | (8.9) | 44 | (8.0) | 111 | (8.6) |
| 3^{b} | 108 | (9.1) | 105 | (9.0) | 96 | (8.5) | 106 | (8.7) |
| 4^{b} | 108 | (9.2) | 106 | (8.9) | 13 | (7.7) | 107 | (8.7) |

^{a}Analyst A
^{b}Analyst B

Free C21 was found to be chemically stable when stored in dark at 5° C., room temperature (RT) and at 40° C. for four weeks. There appears to be a slight decrease in pH when the solution is stored at room temperature or above, but not when it is stored cold.

Peaks in the HPLC chromatogram that correspond to impurities/degradation products were followed by their respective peak area. The total impurity peak area was around 2.5 area% of C21 peak area for the samples stored at 5° C., RT/dark and 40° C.

There is a clear increase in number of impurity peaks in the samples stored at RT/light which suggests that the substance is chemically degraded when exposed to light (at least in the presence of water). Especially, a peak at relative retention time of 0.84 correspond to 6.9 minutes appears under this storage condition.

Precipitation was observed in the sample stored for two and four weeks in RT/light and the samples were therefore filtered (0.45 μm, GHP/Acrodisc) prior to analysis. The comparably low content of 44% and 13%, respectively, may be due to precipitation of C21 which may occur at pHs below 8.0. It is however clear that the decrease in content is also due to formation of degradation products at this storage condition. A number of other impurity peaks were observed by HPLC, which are likely related to the degradation of C21 under this storage condition.

A possible explanation of the pH drop in the sample stored for several weeks in RT/light is that degradation of the substance causes a decrease in pH which in turn sets a limit to the solubility of C21 itself.

The stability of the sodium salt of C21 was also investigated under the same storage conditions. The results are summarised in Table 4 below.

TABLE 4
Storage

| time | Amount of Free C21, % of initial | | | | | | | |
|---|---|---|---|---|---|---|---|---|
| (weeks) | 5° C., dark | | RT, dark | | RT, light | | 40° C., dark | |
| Initial^{a} | 100 | (8.3) | 100 | (8.3) | 100 | (8.3) | 100 | (8.3) |
| 1^{a} | 108 | (8.5) | 115 | (8.6) | 108 | (8.4) | 111 | (8.6) |
| 2^{b} | 113 | (8.4) | 110 | (8.8) | 96 | (8.0) | 111 | (8.5) |
| 3^{b} | 113 | (8.5) | 111 | (8.8) | 72 | (8.3) | 109 | (8.7) |
| 4^{b} | 112 | (8.5) | 112 | (8.2) | 9 | (7.3) | 118 | (8.1) |

^{a}Analyst A
^{b}Analyst B

At the time for analysis of the one week samples, it was noted that the heating cabinet for storage of samples at 40° C. was broken. In view of this, these samples were thereafter kept at room temperature for three days.

As with free C21, the sodium salt is chemically stable after 4 weeks when kept in the dark at all temperatures studied. For the samples stored at RT/light there is a peak occurring at the same relative retention time as observed for free C21. There are also a number of other peaks, which it was thought were related to light induced degradation.

The conclusion is therefore that light-induced degradation occurs in both the sodium salt and free C21.

This presented a significant challenge for development of C21. For any future pharmaceutical product, it is difficult to ensure the complete avoidance of ambient temperatures (or higher), light and moisture at the same time, during drug manufacture, formulation manufacture, packaging, transportation and storage.

It was subsequently decided to formulate C21 as the sodium salt in an aqueous solution in the presence of a carbonate buffer for oral dosing, at concentrations of 0.2 and 10 mg/mL for further pre-clinical and clinical development. Such frozen formulations were found to be chemically stable for 3 months when stored refrigerated in polyethylene terephthalate (PET) bottles and for 36 months when stored in a freezer at −15° C., with no degradation changes in ph or appearance or assay having been observed.

### Example 3

Solubility Study

In view of the issues noted in Example 1 above, as well as the fact that the active ingredient was chemically unstable in the presence of certain dry inert excipients and found to be difficult to compress, dosage forms in the form of dry powdered formulations were considered inappropriate at the relevant time.

Accordingly, the feasibility of incorporating the sodium salt of C21 as a soft gelatin capsule for clinical purpose was evaluated.

In the first instance, formulation studies were conducted to assess the solubility of C21 in pharmaceutically-acceptable lipid-based excipients.

C21 sodium salt (RISE AB, Södertälje, Sweden) was mixed with various potential carriers in the proportions described in Table 5 below (mg of C21 per gram of excipient) and absolute solubility of C21 was determined, two and five days after mixing.

The procedure was carried out by first weighing around 2.955 g of each excipient into a 20 mL headspace vial. Then, 0.045 g of C21 was added to each vial to reach a starting concentration of 15 mg/g. A magnetic stirrer was added into each mixture to stir the dispersion during the entire study (at around 300 rpm).

Solubility was determined at room temperature in general, although some of the excipients listed below (those marked with an asterisk) are solid at room temperature, in which case solubility was determined at 60° C.

The mixtures were observed over time to confirm excipient saturation. In the case of complete C21 solubilization (i.e. no particles were visible), addition of C21 was performed until a maximum API concentration of 100 mg/g was attained.

After confirming that the saturation point had been reached, or after reaching the maximum API concentration of 100 mg/g, sampling of the mixtures were performed after two (T2) and five (T5) days of stirring.

At each tirnepoint, and for each excipient, sampling was performed. The samples were filtered prior to assaying to determine the C21 sodium salt solubility in each excipient.

Two analytical samples were prepared from the filtrate obtain a mean value.

A Waters UPLC Acquity system (CSH C18; 100×2.1 mm×1.7 μm) with a UV and a DED detector was used to quantitatively determine solubility. The following chromatograhpic conditions were applied: (A) mobile phase water with 003% TFA, (6) acetonitrile with 0.03% TFA, with gradient, flow rate 0.5 mL/min, temperature 40° C., run time 24 minutes, injection volume 2 μL at room temperature.

The mean solubilities of C21 sodium salt at specific e points are detailed in Table 5 below and are shown in FIG. 1.

Excipients are grouped by chemical class to better understand the solubility results obtained. It should be noted that Gelucires are a group of vehicles acquired from blends of mono, di- and triglycerides with PEG esters of unsaturated fats. Gelucire 43/01 is a hydrophobic grade that contains glycerides only.

TABLE 5

| | | C21 Mean | |
|---|---|---|---|
| | | Solubility | |
| | Load | T2 | T5 |
| Excipient (Generic | of C21 | Assay | Assay |
| and/or Commercial Name) | (mg/g) | (mg/g) | (mg/g) |

Triacylglycerols

| | | | | |
|---|---|---|---|---|
| 1 | Refined sesame oil (Henry Lamotte) | 15.5 | 0.2 | 0.2 |
| 2 | Refined corn oil (Henry Lamotte) | 15.4 | 0.0 | 0.0 |
| 3 | Soya oil (Henry Lamotte) | 15.5 | 0.0 | 0.0 |
| 4 | Medium chain triglycerides | 15.3 | 0.0 | 0.0 |
| | (Miglyol 812N; Cremer Oleo) | | | |
| 5 | Gelucire 43/01 (Gattefosse)* | 15.5 | 0.0 | 0.0 |
| 6 | Triacetin (Kollisolv GTA; BASF) | 15.4 | 0.2 | 0.3 |

Triacyl Sorbitans

| | | | | |
|---|---|---|---|---|
| 7 | Sorbitan trioleate (Span 85; Croda) | 13.6 | 15.1 | 12.1 |

Monoacyl Propylene Glycols

| | | | | |
|---|---|---|---|---|
| 8 | Propylene glycol monolaurate | 13.6 | 12.4 | 12.4 |
| | (Lauroglycol 90; Gattefosse) | | | |
| 9 | Propylene glycol monocaprylate | 13.8 | 7.1 | 7.1 |
| | (Capryol PGMC; Gattefosse) | | | |

Polyoxylglycerides

| | | | | |
|---|---|---|---|---|
| 10 | Linoleoyl polyoxyl-6-glycerides | 30.6 | 22.0 | 21.1 |
| | (Labrafil M 2125 CS; Gattefosse) | | | |
| 11 | Oleoyl polyoxyl-6-glycerides | 31.4 | 19.5 | 20.1 |
| | (Labrafil M 1944 CS; Gattefosse) | | | |
| 12 | Lauryl polyoxyl-6-glycerides | 15.6 | 13.9 | 13.5 |
| | (Labrafil M 2130 CS; Gattefosse)* | | | |

Monoacylglycerols and Monoacyl Sorbitans

| | | | | |
|---|---|---|---|---|
| 13 | Glyceryl monolinoleate | 80.3 | 61.4 | 60.4 |
| | (Maisine CC; Gattefosse) | | | |
| 14 | Sorbitan monooleate | 65.1 | 61.6 | 61.5 |
| | (Montane 80; SEPPIC) | | | |
| 15 | Glyceryl monooleate (Peceol; | 114.5 | 82.3 | 82.2 |
| | Gattefosse) | | | |

Hydrophilic Surfactants

| | | | | |
|---|---|---|---|---|
| 16 | Gelucire 44/14 (Gattefosse)* | 80.2 | 44.1 | 43.9 |
| 17 | Gelucire 50/13 (Gattefosse)* | <96.5 | N/A¹ | N/A¹ |
| 18 | Polyoxyethylene (20) sorbitan | 111.5 | 85.2 | 84.8 |
| | monooleate (Tween 80; Croda) | | | |

Simulated Intestinal Fluids

| | | | | |
|---|---|---|---|---|
| 19 | Fasted Simulated Intestinal | 15.1 | 10.4 | 10.4 |
| | Fluid (FaSSIF)² | | | |
| 20 | Fed Simulated Intestinal | 15.0 | 0.3 | 0.6 |
| | Fluid (FeSSIF)² | | | |

¹Precipitation occurred after filtration into the solvent, which made it impossible to test the samples
²Recipe described in Biorelevant.com followed

It is clear from Table 5 and FIG. 1 that C21 solubilization is highly dependent on the number of free hydroxyl groups that are present in an excipient, and also that it is essentially insoluble in triglyceride-based excipients, which insolubility is independent of carbon chain length and the degree of unsaturation of the fatty acid component.

Sorbitan trioleate and monoesters of the propylene glycol family were also considered to be of interest, because they demonstrated poor API solubilization properties. Additionally, Labrafil M2130CS, from the mono-di-tri-glycerides family, was considered to be of interest as solubility of around 14 mg/g was achieved, although at 60° C. However, as this is a solid excipient at room temperature, solubility at room temperature was expected to be lower.

### Example 4

Compatibility Studies

Experiments were then performed to assess the chemical compatibility of C21 with selected pharmaceutically-acceptable lipid-based ingredients (some, but not all, of which were also studied in Example 3 above), as well as the main soft shell gelatin capsule components, under accelerated conditions.

The compatibility study was performed by storing at 40° C. and 75% RH for eight weeks in a climatic chamber (Weiss Technik), during which an analysis of impurities formed was performed after four (T4) and eight (T8) weeks.

An additional experiment was performed by storing for eight weeks at room temperature in the laboratory (with controlled temperature and humidity).

Testing of C21 sodium salt in the absence of excipients was performed as a reference.

The composition of the mixtures that were analysed are presented in Table 6 below. In Table 6, the generic names and the sources of the various excipients are the same as presented above, e.g. in Table 5.

TABLE 6

| | | Composition (%) | | |
|---|---|---|---|---|
| Ref. | Sample | C21 | Excipients | Water |
| A.0 | C21 | 100 | 0 | |
| A.1 | C21/Refined sesame oil | 1 | 99 | 0 |
| A.2 | C21/Refined corn oil | 1 | 99 | 0 |
| A.3 | C21/Soya oil | 1 | 99 | 0 |
| A.4 | C21/Miglyol 812N | 1 | 99 | 0 |
| A.5 | C21/Kollisolv GTA | 1 | 99 | 0 |
| A.6 | C21/Miglyol 812N/Gelucire | 1 | 99 | 0 |
| | 43/01 (95:5) | | | |
| A.7 | C21/Miglyol 812N/HVO | 1 | 99 | 0 |
| | type II¹ (95:5) | | | |
| A.8 | C21/Miglyol 812N/Aerosil | 1 | 99 | 0 |
| | R97² (95:5) | | | |
| A.9 | C21/Span 85 | 1 | 99 | 0 |
| A.10 | C21/Lauroglycol 90 | 1 | 99 | 0 |
| A.11 | C21/Capryol PGMC | 1 | 99 | 0 |
| A.12 | C21/Labrafil M 2130 CS | 1 | 99 | 0 |
| A.13 | C21/Glycerol³ | 1 | 99 | 0 |
| A.14 | C21/Anidrisorb 85/70⁴ | 1 | 99 | 0 |
| A.15 | C21/Gelatin⁴ (5% in water) | 1 | 99 | 0 |
| A.16 | C21/Water | 1 | 99 | 0 |
| B.1 | C21/Glycerol/Water | 1 | 89 | 10 |
| C.1 | Refined sesame oil | 0 | 100 | 0 |
| C.2 | Refined corn oil | 0 | 100 | 0 |
| C.3 | Soya oil | 0 | 100 | 0 |
| C.4 | Miglyol 812N | 0 | 100 | 0 |
| C.5 | Kollisolv GTA | 0 | 100 | 0 |
| C.6 | Miglyol 812N/Gelucire 43/01 | 0 | 100 | 0 |
| | (95:5) | | | |
| C.7 | Miglyol 812N/HVO type II (95:5) | 0 | 100 | 0 |
| C.8 | Miglyol 812N/Aerosil R972 (95:5) | 0 | 100 | 0 |
| C.9 | Span 85 | 0 | 100 | 0 |
| C.10 | Lauroglycol 90 | 0 | 100 | 0 |
| C.11 | Capryol PGMC | 0 | 100 | 0 |
| C.12 | Labrafil M2130CS | 0 | 100 | 0 |
| C.13 | Glycerol | 0 | 100 | 0 |
| C.14 | Anidrisorb | 0 | 100 | 0 |
| C.15 | Gelatin (5% in water) | 0 | 100 | 0 |
| C.16 | Water | 0 | 100 | 0 |
| D.1 | Glycerol/Water | 0 | 90 | 10 |

¹Hydrotreated Vegetable Oil (Aarhus Karlshamn)
²Hyrophobic fumed silica (Evonik; fumed silica treated with dimethyidichlorosilane)
³Gelatin (Gelita), glycerol (Cremer Oleo), Anidrisorb 85/70 (Roquette; sorbitol, mannitol, sorbitan, hydrogenation products of partly hydrolyzed starch; source?) and water (distilled) are the components of soft gelatin capsules

Samples A0 to A16 and B1 were prepared in 20 mL glass vials, with two preparations being prepared for each time point. Samples C1 to C16 and D1 were prepared in 20 mL glass vials, with one preparation for each time point.

Assay and impurity evaluations were made using the same Waters UPLC Acquity system and essentially the same chromatographic conditions as described in Example 3 above.

The impurity analysis is summarized in Table 7 below, in which C21 assay values (I) and impurity values (II) are presented as % recovery and represent the average value obtained for each mixture on the two sample preparations.

TABLE 7

| | | | T4 | | T8 | | | |
|---|---|---|---|---|---|---|---|---|
| | T0 | | 40° C. | | 40° C. | | Ambient temp. | |
| Ref. | I | II | I | II | I | II | I | II |
| A.0 | 98.2 | 0.24 | 102.2 | 0.23 | 101.3 | 0.25 | 102.0 | 0.24 |
| A.1 | | | 95.4 | 0.23 | 95.6 | 0.28 | 99.4 | 0.24 |
| A.2 | | | 97.6 | 0.47 | 97.2 | 0.27 | 95.5* | 0.24 |
| A.3 | | | 96.9 | 0.23 | 99.2 | 0.25 | 98.7 | 0.23 |
| A.4 | | | 97.6 | 0.23 | 99.1 | 0.23 | 99.3 | 0.25 |
| A.5 | | | 97.8 | 0.26 | 97.6 | 0.27 | 102.1 | 0.24 |
| A.6 | | | 96.7 | 0.24 | 99.0 | 0.26 | 98.0 | 0.24 |
| A.7 | | | 92.3 | 0.24 | 93.9 | 0.25 | 96.6 | 0.23 |
| A.8 | | | 93.5 | 0.65 | 98.8 | 0.53 | 96.1 | 0.26 |
| A.9 | | | 25.0* | 76.0 | 2.9 | 95.8 | 73.2 | 16.0 |
| A.10 | | | 83.2* | 15.5 | 67.2 | 30.5 | 95.2 | 2.70 |
| A.11 | | | 86.9* | 9.14 | 61.7* | 24.9 | 94.7 | 1.90 |
| A.12 | | | 73.5* | 17.4 | 74.2 | 29.1 | 95.1 | 1.57 |
| A.13 | | | 93.2 | 0.48 | 81.9* | 1.45 | 98.6 | 0.25 |
| A.14 | | | 94.1 | 1.80 | 80.1* | 2.30 | 95.9 | 0.28 |
| A.15 | | | 87.8 | 1.64 | 92.6* | 2.60 | 94.1* | 0.51 |
| A.16 | | | 97.1* | 0.71 | 98.4 | 1.13 | 98.5 | 1.16 |
| B.1 | | | 94.4 | 1.08 | 93.2 | 2.35 | 98.5 | 0.25 |

*Individual preparation results reported, as outlier results were discarded

These compatibility study results show that the API is stable at least 8 weeks at 40° C. in triglycerides ingredients (see results for Samples A1 to A5), and that this is independent of aliphatic chain length.

Equivalent API stability is observed with long chain triglycerides (e.g. refined sesame oil, refined corn oil or soya oil) versus medium or short chain triglycerides (e.g. Miglyol 812N and Kollisolv GTA).

Addition of thickening agents is also not expected to have an impact on API stability. Mixtures of Miglyol and hydrophobic thickening agents also presented good stability results (see results for Samples A6 to A8), although the result for Sample A8 (Miglyol/Aerosil R972) was less favourable.

Lipophilic surfactants strongly degrade the API with a significant decrease of API assay results and an increase of level of impurities observed after only 4 weeks of storage at 40° C. (see results for Samples A9 to A12).

Finally, in the case of the soft gelatin capsule shell components (A13-A15 and B1), a slight increase of impurity level is observed at T4 weeks and confirmed after 8 weeks. The API seems to be more stable in glycerol than in Anidrisorb 85/70, but the addition of water leads to similar level of impurity than those observed with Anidrisorb alone.

However, the examined mixture of glycerol and water comprised 10% water, which represents the worst case scenario for a putative soft gel capsule and water uptake. Thus, glycerol remains the most promising plasticizer to implement to limit API degradation.

A slight increase of impurity level is also observed with the mixture API/gelatin (with 5% of water).

Nevertheless, this study documents results and conditions that are worstcase scenarios for the capsule ingredients.

Furthermore, during the study, C21 sodium salt was in solution in the shell ingredient, which is a situation that will not occur in accordance with the dosage form of the invention, given that in such a finished product, active ingredient will be suspended in a hydrophobic oil-based carrier, in which it is essentially insoluble, which will result in very limited interaction with the capsule shell.

### Example 5

Dosage Form of the Invention

A rotary die encapsulation process (see, for example, Pharmaceutics, The Science and Manufacture of Medicines, Aulton etal. (eds.) 4^{th} edition (2013)) is employed to make dosage forms of the invention. Appropriate equipment is available from, for example, Sinagel Technology, China.

C21 sodium salt is dispersed in one or more of the triglyceride media mentioned in Examples 3 or 4 above to give a suspension.

A thickening agent (Miglyol 812) is added to the suspension to increase the viscosity and reduce sedimentation of the solid C21 salt particles, leading to a fully heterogeneous suspension.

After this, gelatin is heated to 60° C. and a plasticizer (glycerol) and a small amount of water (not more than about 5%) is added to the molten gelatine mass.

The molten gelatin is allowed to flow from a tank containing it to two heated pipes and through two heated spreader boxes, onto two large, cooled casting drums maintained at 16-20° C. Two flat solid ribbons of gel are formed, which are fed between mineral oil lubricated rollers into the encapsulation mechanism.

At the same time, the suspension of active ingredient is allowed to flow from a product material tank to a multi-plunger positive displacement filling pump. Accurately metered volumes of the liquid fill material are injected through the wedge (heated to 37-40° C.) between the gelatin ribbons as they pass between the die rolls.

The injection of liquid forces the gelatin to expand into the pockets of the dies and governs the size and shape of the capsules. The ribbon continues to flow past the heated wedge and is pressed between the die rolls where the capsule halves are sealed together by the application of heat (37-40° C.) and pressure.

The capsules are cut out automatically from the gelatine ribbons by the dies, and are transported through a wash to remove surface lubricating oil.

The capsules are then passed through a rotating basket, infra-red dryer and are then spread onto trays to complete the drying process in a tunnel corridor using air at a relative humidity of approximately 20%.

Thereafter the capsules are inspected for quality, washed again if necessary, graded according to specification and are packaged in for distribution.

1. A pharmaceutical dosage form that is suitable for peroral administration to the gastrointestinal tract, which dosage form comprises a pharmaceutical composition in the form of a heterogeneous mixture comprising solid particles of N-butyloxycarbonyl-3-(4-imidazol-1-ylmethylphenyl)-5-iso-butylthiophene-2-sulfonamide, or a pharmaceutically-acceptable salt thereof, suspended in a pharmaceutically-acceptable, hydrophobic, lipid-based carrier in which N-butyloxycarbonyl-3-(4-imidazol-1-ylmethylphenyl)-5-iso-butylthiophene-2-sulfonamide or salt thereof is essentially insoluble, which composition is contained within a capsule that is suitable for such peroral administration.

2. A dosage form as claimed in claim 1, wherein the capsule is a soft-shell, single-piece capsule.

3. A dosage form as claimed in claim 2, wherein the capsule is a soft gelatin capsule.

4. A dosage form as claimed in any one of the preceding claims, wherein the lipid-based carrier is mainly comprised of triglycerides.

5. A dosage form as claimed in claim 4, wherein the carrier system comprises at least about 90% triglycerides.

6. A dosage form as claimed in claim 4 or claim 5, wherein the triglycerides comprise one or more fatty acids selected from the group caproic acid, caprylic acid, capric acid, auric acid, myristic acid, palrnitic acid, stearic acid, oleic acid, ricinoleic acid, linoleic acid, linolenic acid, eicosenoic acid, behenic acid and erucic acid.

7. A dosage form as claimed in any one of claims 4 to 6, wherein the triglyceride is a naturally-occurring oil or fat.

8. A dosage form as claimed in claim 7, wherein the naturally-occurring oil is selected from the group sesame oil, corn oil, palm kernel oil, coconut oil or soya oil.

9. A dosage form as claimed in any one of claims 4 to 6, wherein the triglycerides are in a semi-synthetic or a synthetic lipid-based carrier system.

10. A dosage form as claimed in claim 9, wherein the lipid-based carrier system is selected from the group short chain triglycerides or medium chain triglycerides.

11. A dosage form as claimed in claim 10, wherein the lipid-based carrier system is selected from triacetin or Miglyol 812N.

12. A dosage form as claimed in any one of the preceding claims that is essentially water-free.

13. A dosage form as claimed in any one of the preceding claims wherein the particles of N-butyloxycarbonyl-3-(4-imidazol-1-ylmethylphenyl)-5-iso-butyl-thiophene-2-sulfonamide or pharmaceutically-acceptable salt thereof have a weight- and/or a volume-based mean diameter that is no more than about 50 μm.

14. A dosage form as claimed in any one of the preceding claims wherein the suspension further comprises a thickening agent.

15. A dosage form as claimed in any one of the preceding claims wherein the pharmaceutically-acceptable salt of N-butyloxycarbonyl-3-(4-imidazol-1-ylmethyl-phenyl)-5-iso-butylthiophene-2-sulfonamide is a sodium salt.

16. A process for the production of a suspension as defined in any one of the preceding claims, which process comprises:
(a) mixing particles of N-butyloxycarbonyl-3-(4-imidazol-1-ylmethyl-phenyl)-5-iso-butylthiophene-2-sulfonamide or pharmaceutically-acceptable salt thereof with the lipid-based carrier, to form the suspension; and
(b) loading the suspension from step (a) into a capsule that is suitable for peroral administration.

17. A dosage form obtainable by a process as defined in claim 16.

18. A dosage form as defined in any one of the claim 1 to 15 or 17 for use in the treatment of an interstitial lung disease.

19. The use of a dosage form as defined in any one of the claim 1 to 15 or 17 for the manufacture of a medicament for the treatment of an interstitial lung disease.

20. A method of treatment of an interstitial lung disease, which method comprises the administration of a dosage form as defined in any one of the claim 1 to 15 or 17 to a patient in need of such treatment.

21. A dosage form for use as defined in claim 18, a use as defined in claim 19, or a method of treatment as defined in claim 20, wherein the interstitial lung disease is idiopathic pulmonary fibrosis.

22. A dosage form for use as defined in claim 18, a use as defined in claim 19, or a method of treatment as defined in claim 20, wherein the interstitial lung disease is sarcoidosis.

23. A dosage form as defined in any one of the claim 1 to 15 or 17 for use in the treatment of respiratory virus-induced tissue damage.

24. The use of a dosage form as defined in any one of the claim 1 to 15 or 17 for the manufacture of a medicament for the treatment of respiratory virus-induced tissue damage.

25. A method of treatment of respiratory virus-induced tissue damage, which method comprises the administration of a dosage form as defined in any one of the claim 1 to 15 or 17 to a patient in need of such treatment.

26. A dosage form for use as defined in claim 23, a use as defined in claim 24, or a method of treatment as defined in claim 25, wherein the damage comprises injury and/or dysfunction of the mucosal tissue of the respiratory tract that is caused by a respiratory virus.

27. A dosage form for use, a use or a method of treatment as claimed in claim 26, wherein the respiratory virus is a coronavirus or is an influenza virus.

28. A dosage form for use, a use or a method of treatment as claimed in claim 27, wherein the respiratory virus is severe acute respiratory syndrome coronavirus 2.

29. A dosage form for use, a use or a method of treatment as claimed in any one of claims 23 to 28 (as appropriate), wherein the treatment includes treatment of the symptoms of the disease that is being, or has been, caused by the virus.

30. A dosage form for use, a use or a method of treatment as claimed in claim 29, wherein the symptoms of the damage or the disease include one or more of couch, dyspnea, respiratory distress, respiratory failure, pneumonia, fibrosis in one or more internal organs selected from the lungs, the heart and/or the kidneys.

31. A dosage form for use, a use, or a method of treatment as defined in any lone of claims 18 to 30 (as appropriate), wherein the treatment includes prevention of morbidity and/or mortality in the relevant condition.

32. A dosage form for use, a use, or a method of treatment as defined in any lone of claims 18 to 31 (as appropriate), wherein the dosage form is administered by the peroral route.
